(19) United States
(12) Patent Application Publication
(10) Pub. No.: US 2018/0310859 A1
(43) Pub. Date: Nov. 1, 2018

(54) Range of Motion Device for Joint Angle Measurement

(71) Applicant: WiSys Technology Foundation, Inc., Madison, WI (US)

(72) Inventors: Jacob Knabe, Rockford, IL (US); Joshua Inglett, Elkhorn, WI (US); Kyle Valenza, Wind Lake, WI (US)

(21) Appl. No.: 15/963,726

(22) Filed: Apr. 26, 2018

Related U.S. Application Data
(60) Provisional application No. 62/490,265, filed on Apr. 26, 2017.

(51) Int. Cl. A61B 5/107; A61B 5/00; A61B 5/11

(52) U.S. Cl. CPC A61B 5/1071; A61B 5/0022; A61B 5/1121; A61B 2562/166; A61B 5/1126; A61B 2562/222; A61B 2562/164; A61B 5/6813

(57) ABSTRACT

The present invention provides a range of motion measurement device using electroactive polymer (EAP) sensors to derive digital measurement angles. The EAP sensors may be attached to a stationary arm and rotating arm of the range of motion measurement device to detect a percentage stretched of the EAP sensors after measuring movement is performed. The percentage stretched of the EAP sensors based upon the law of cosines may be used with a transfer function in order to determine the range of motion angle of the joint.

### CROSS REFERENCE TO RELATED APPLICATION

This application claims the benefit of U.S. provisional application 62/490,265 filed Apr. 26, 2017 and hereby incorporated by reference.

### STATEMENT REGARDING FEDERALLY SPONSORED RESEARCH OR DEVELOPMENT

### Background of the Invention

The present invention relates to angle measuring devices and in particular to a device used to record the range of motion of a patient's joint such as the knee or elbow.

Angle measurements are frequently used in the field of physical therapy and occupational therapy. A body's joints have certain degrees of freedom of movement. Decreased range of motion caused by abnormal growths in or around the joint, damage to the joint, tightness of surrounding muscles, or pain due to an underlying condition, may be an indication that medical intervention is needed. Specifically, range of motion measurement is commonly needed in people recovering from surgery for knee replacement, arthritis patients, cerebral palsy patients, and special needs patients. These types of angle measurements may be used to diagnose and track the progress of rehabilitation in a patient with decreased range of motion.

A goniometer is a commonly known instrument used to measure the range of motion of joint angles of the body. The goniometer is a device having a proximal or stationary arm extending from a circular disk. The stationary arm and circular disk do not move during joint measurement. The circular disk has a discrete scale for reading the angle measurement of a distal or rotating arm that rotates about a center axis of the circular disk. For example, in range of motion measurement of the knee joint, the center axis of the circular disk (i.e., point of rotation) is placed at the lateral epicondyle of the femur while the stationary arm is lined up with the greater trochanter of the femur. Then the rotating arm is lined up with the rotated lateral malleolus of the fibula and an angle measurement is taken using the degree scale on the circular disk of the goniometer.

Accuracy of the angle measurement using the goniometer is low, with errors typically between five and ten degrees. Often these errors arise from issues with measurements being in between measurement markings or from inconsistent technique between clinicians.

### SUMMARY OF THE INVENTION

The present invention provides a range of motion measurement device using electroactive polymer (EAP) sensors to derive digital measurement angles. The EAP sensors may be attached to a “stationary” arm and “rotating” arm of the range of motion measurement device to detect a percentage stretched of the EAP sensors after joint movement is performed. The percentage stretched of the EAP sensors may be used with a transfer function based upon the law of cosines in order to determine the range of motion of the joint.

It is thus a feature of one embodiment of the present invention to utilize EAP sensor technology in the development of more accurate angle measurement.

The EAP sensor may be installed by the patient on their own body, and maneuvered and secured by the straps to provide proper alignment.

It is thus a feature of one embodiment of the present invention to allow patients to monitor their own progress while at home. This eliminates the number of trips that the patient needs to take to a clinic or hospital. The measurement data may be transmitted to a remote clinician.

The present invention provides a medical goniometer having a first patient support attached to a first side of a patient's joint; a second patient support tangibly attached to the first patient support and hingably attached to an opposite second side of the patient's joint so that the first and second supports pivot about a hinge axis; an elastomeric sensor providing electrical signal as a function of distortion of the sensor connected between the first patient support and the second patient support to be distorted by the pivoting; and electrical leads attached to the elastomeric sensor to provide an output signal indicating an angle between the first patient support and second patient support.

It is thus a feature of one embodiment of the present invention to increase accuracy of human joint range of motion measurements.

The elastomeric sensor may provide a change in capacitance with distortion of the elastomeric sensor. The elastomeric sensor may be two stretchable electrodes separated by an elastomer. The two stretchable electrodes may be carbon ink electrodes and the elastomer may be a silicone dielectric polymer.

It is thus a feature of one embodiment of the present invention to provide a sensor that is easy to clean.

The electrical leads may be attached to the elastomeric sensor at one end.

It is thus a feature of one embodiment of the present invention to keep wires from becoming tangled or interfere during joint movement.

The elastomeric sensor may comprise of first and second sensors.

It is thus a feature of one embodiment of the present invention to reduce non-linearity of the transfer function.

Each of the first and second sensors may be pivotably connected to the first patient support and second patient support about respective pivot axes. The pivot axes and hinge axis may be substantially aligned in parallel. The first and second sensors may have a shared pivot axis on the second patient support.

It is thus a feature of one embodiment of the present invention to use two inter-related sensor measurements for improved accuracy.

The distortion of the sensor may be a change in length.

The first patient support and second patient support may further comprise support straps adapted to wrap around the patient's body or limb at the first side and second side of the patient's joint.

It is thus a feature of one embodiment of the present invention to secure the patient supports to the patient during motion so the sensors do not deviate in position.

The medical goniometer may further comprise a transmitter transmitting the output signal to a computer. The computer may be a mobile device.

It is thus a feature of one embodiment of the present invention to allow the measurement to be performed by the patient and transmitted to a remote clinician for further processing.

The present invention also provides an angle measurement device having a first support; a second support pivotable with respect to the first support about a hinge axis and defining an angle of rotation between the first support and the second support; a first elastomeric sensor providing electrical signal as a function of distortion of the sensor connected between the first support and the second support to be distorted by the pivoting; a second elastomeric sensor providing electrical signal as a function of distortion of the sensor connected between the first patient support and the second patient support to be distorted by the pivoting; and electrical leads attached to the elastomeric sensor to provide an output signal indicating an angle between the first support and second support.

The angle measurement device may further comprise a computer processor receiving the output signal and executing a stored program to output the angle of rotation between the first support and the second support.

The first support may have a first section and a second section disposed at an angle wherein the first elastomeric sensor is attached to the first section and the second elastomeric sensor is attached to the second section. The angle may be between 130 and 140 degrees.

The present invention also provides a method of measuring an angle of rotation comprising: providing an angle measurement device having a first patient support adapted to attach to a first side of a patient's joint; a second patient support tangibly attached to the first patient support and hingably attached to an opposite second side of the patient's joint so that the first and second supports pivot about a hinge axis; an elastomeric sensor providing electrical signal as a function of distortion of the sensor connected between the first patient support and the second patient support to be distorted by the pivoting; and electrical leads attached to the elastomeric sensor to provide an output signal indicating an angle between the first patient support and second patient support; attaching the first support to a first side of a patient's joint; attaching the second support to a second side of the patient's joint; and outputting the angle of rotation defined between the first support and the second support

These particular objects and advantages may apply to only some embodiments falling within the claims and thus do not define the scope of the invention.

### BRIEF DESCRIPTION OF THE DRAWINGS

FIG. 1 is a perspective view of a patient laying prone on a table with a simplified range of motion measurement device strapped to a leg surrounding the knee joint according to one embodiment of the present invention;

FIG. 2 is an enlarged perspective view of the range of motion measurement device installed on the patient's leg of FIG. 1 showing the electroactive polymer sensors installed between the stationary arm and rotating arm;

FIG. 3 is a schematic representation of the axes of the range of motion device using electroactive polymer sensors to calculate a range of motion angle of the patient's leg;

FIG. 4 is a graphical representation of the transfer function used to calculate the range of motion angle based upon an inputted percentage stretch of the electroactive polymer sensors; and

FIG. 5 is an enlarged side elevation view of the electroactive polymer sensor.

### DETAILED DESCRIPTION OF THE PREFERRED EMBODIMENT

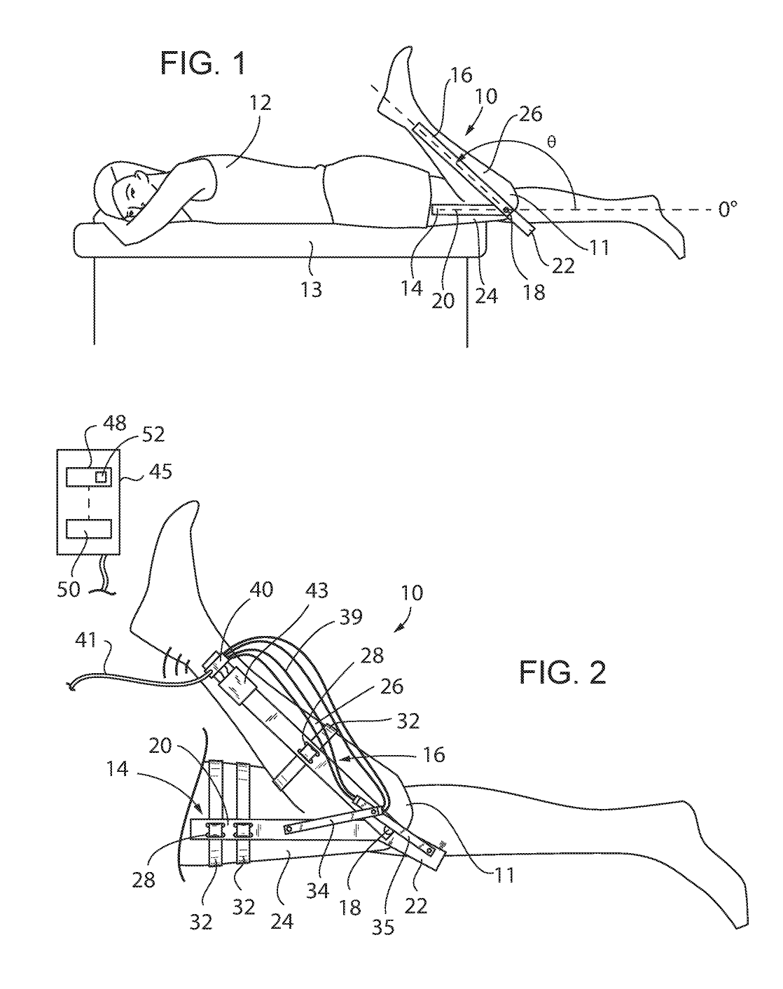

Referring now to FIG. 1, a range of motion measurement device 10 is designed to be attached to a human patient 12 for measurement of a patient's joint angles of the body. Although the range of motion measurement device 10 is shown and described to be used with measurement of range of motion of a patient's knee joint 11, the range of motion measurement device 10 may also be used to measure any joint angle of the body such as a patient's hipbone, knee joint, ankle bone, elbow joint, etc.

As shown in FIGS. 1 and 2, the patient 12 may lie prone, face down on a bed or table 13 in order to measure the joint angle of the patient's knee joint 11 when flexed such that the patient's upper leg 24 (above the knee joint 11) remains on the bed or table 13 and the patient's lower leg 26 (below the knee joint 11) is rotated upward with respect to the top surface of the bed or table 13. The patient 12 may be positioned such that the lower leg 26 hangs off the back of the bed or table 13.

The range of motion measurement device 10 may include a stationary arm 14 (attached to a non-moving part of the body, e.g., the upper leg 24) that does not rotate and a rotating arm 16 (attached to a moving part of the body, e.g., the lower leg 26) that rotates with respect to the stationary arm 14 about a hinge axis 18 of the stationary arm 14 prior to joint measurement.

The stationary arm 14 may be a long segment 20 and a short segment 22 fixedly connected at an angle that is between 130 and 140 degrees or approximately 135 degrees to form a V-shape. The long segment 20 is a rectangular straight strip portion resembling a measurement ruler and having a width of approximately 1 inch and a length between 3 inches and 12 inches. The long segment 20 is joined at its connection end 21 to a connection end 23 of the short segment 22 that is also a rectangular straight strip segment resembling a measurement ruler and having a width of approximately 1 inch and a length of approximately 3 inches. The long segment 20 may be approximately the same length or preferably longer than the short segment 22.

The stationary arm 14 may be attached to the patient's upper leg 24 and aligned with the greater trochanter of the femur while the short segment 22 may extend downward toward the patient's feet. In this respect, the stationary arm 14 may take an inverted V-shape or downward V-shape with the short segment 22 extending downward off a side or back of the bed or table 13 when the patient 12 is lying prone, face down (as seen in FIGS. 1-2). The length of the long segment 20 may generally correspond to or be shorter than an average length of the patient's upper leg 24.

The rotating arm 16 may be a rectangular straight strip resembling a measurement ruler having a width of approximately 1 inch and a length between 3 inches and 12 inches. The rotating arm 16 may be pivotably attached at a lower connection end 25 of the rotating arm 16 to the stationary arm 14 at the hinge axis 18 proximate the connection ends 21, 23 of the long segment 20 and short segment 22 respectively.

The rotating arm 16 may be attached to the patient's lower leg 26 and aligned with the lateral malleolus of the fibula. The length of the rotating arm 16 may generally correspond to or be shorter than an average length of the patient's lower leg 26.

The hinge axis 18 may be defined as a point of rotation between the stationary arm 14 and rotating arm 16 and may generally be aligned with the patient's lateral epicondyle of the femur.

Referring to FIG. 2, the stationary arm 14 and rotating arm 16 may include oblong or rectangular slots or holes 28 that allow belts or straps 32 having fasteners such as clasps or Velcro to extend through the holes 28 and around the patient's upper leg 24 and patient's lower leg 26 to secure the stationary arm 14 and rotating arm 16 to the patient's body without sliding movement therebetween. The straps 32 may generally extend perpendicular to the stationary arm 14 and rotating arm 16.

The stationary arm 14 and rotating arm 16 may be constructed of acrylic, metal, or plastic material, or the like which is durable and rigid. The corners of the stationary arm 14 and rotating arm 16 may be rounded to prevent injury to the patient 12 during movement.

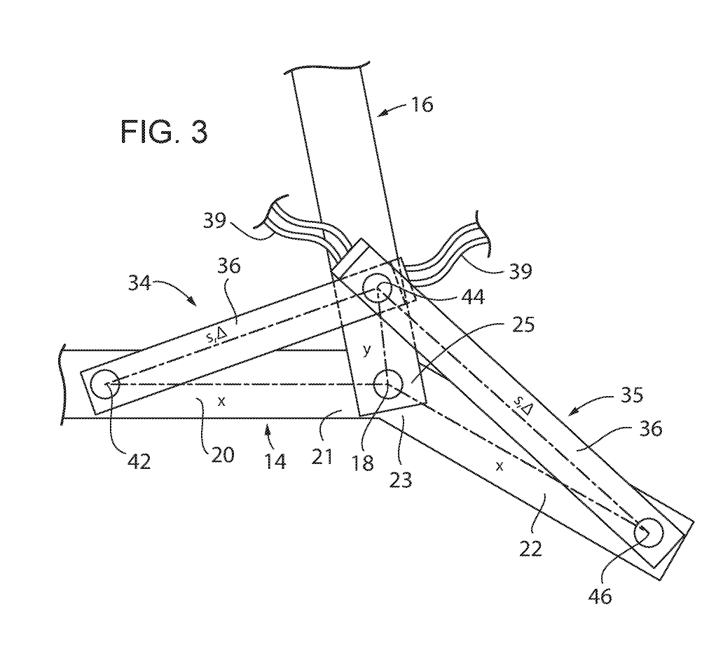

Referring to FIGS. 2 and 3, the range of motion measurement device 10 may further include electroactive polymer (EAP) sensors 34, 35 attached at opposite ends to the stationary arm 14 and rotating arm 16, respectively, to measure an angle of rotation θ of the patient's knee joint 11 after flexure.

A first EAP sensor 34 may be extend between a pivot axis 42 on the long segment 20 of the stationary arm 14 and a pivot axis 44 on the rotating arm 16. A second EAP sensor 35 may extend between a pivot axis 46 on the short segment 22 of the stationary arm 14 and the same pivot axis 44 on the rotating arm 16 also attaching the first EAP sensor 34. The pivot axes 42, 44, 46 are generally located distal to the hinge axis 18 such that the pivot axes 42, 44, 46 are spaced from the hinge axis 18. The pivot axes 42, 44, 46 may be defined by bolts 47 extending along the axes through the stationary arm 14, rotating arm 16, and EAP sensors 34, 35 (see FIG. 5). The axes of the pivot axes 42, 44, 46 may be aligned in parallel and may also be aligned in parallel with the hinge axis 18.

Referring to FIG. 5, the EAP sensors 34, 35 may comprise of a flexible dielectric polymer 38 that acts as a displacement-to-capacitance transducer by exhibiting a change in size or shape when stimulated by an electric field. When the polymer 38 is stretched thin and expands, the capacitance increases. This characteristic of the polymer 38 may be used to measure strain on the first and second EAP sensor 34, 35. The EAP sensors 34, 35 are reversibly stretchable to completely return to its un-stretched state for repeated strain measurement.

The EAP sensors 34, 35 may each include a generally rectangular EAP film 36 that is made of two parallel opposed electrodes 37 separated by a polymer 38 such as an elastomer, for example, two stretchable carbon ink electrodes 37 separated by a silicone dielectric polymer 38 disposed therebetween. As the EAP film 36 is stretched or strained, the polymer 38 expands or compresses within the area changing the capacitance. The capacitance measurement of the EAP film 36 is then delivered as an output signal through electrical leads 39 coupled to each of the electrodes 37 of the EAP film 36. The EAP sensors 35, 35 may also be configured to output a compressive strain, or length change signal, e.g., as a raw number or as a percentage change, of the EAP film 36.

The EAP sensors 34, 35 of this type are commercially available from, e.g., Parker Hannifin Corporation of Cleveland, Ohio, sold as “Parker EAP Sensors Evaluation Kit” and used in connection with Parker's SensiNODE™ mobile application software. See

http://ph.parker.com/us/en/electroactive-polymer-technology-monitors-movement-and-stretch -eap-sensor-evaluation-kits, and
https://promo.parker.com/parkerimages/promosite/ArtificialMuscle/UNITED%20STATES/Abo ut%20Electroactive%20Polymer/PDF/EAP-Bulletin.pdf, both of which are hereby incorporated by reference.

Referring again to FIGS. 2 and 3, the electrical leads 39 may transmit the output signal to a transmitter 40. The transmitter 40 may then transmit the information to an external computer 45, for example a mobile device, for future evaluation. The transmitter 40 may transmit information to the external computer 45 wirelessly or through a hard wired connection, such as a USB cable 41.

The EAP sensors 34, 35 and transmitter 40 may be powered by a battery 43 through a wireless or hard wired connection. The transmitter 40 and battery 43 may be supported by the range of motion measurement device 10, for example, attached to the stationary arm 14 or rotating arm 16.

Referring to FIGS. 1-3, in use, the prone, downward facing patient 12 may flex their knee 11 to define an angle of rotation θ between the stationary arm 14 and the rotating arm 16. As the rotating arm 16 pivots or rotates with respect to the rotating arm 16 about the hinge axis 18, the first EAP sensor 34 shortens while the second EAP sensors 35 elongates changing the stretch of the respective EAP films 36 of the first and second EAP sensors 34, 35. For example, as the EAP film 36 of the first EAP sensors 34 expands, the EAP film 36 of the second EAP sensor 35 shortens. Conversely, as the EAP film 36 of the first EAP sensor 34 shortens, the EAP film 36 of the second EAP sensor 35 stretches. The stretch of the EAP sensors 34, 35 causes a change in capacitance between the two electrodes 37.

The first and second EAP sensors 34, 35 may be configured to receive the change in capacitance and transmit compressive strain related output data, e.g., a raw number or percentage stretch. For example, the EAP sensors 34, 35 may output a changed length divided by the original length, or a change in length divided by the original length of the EAP film 36. The compressive strain related data from the EAP sensor 34, 35 may then be used to calculate the angle of rotation θ of the patient's knee joint 11, as further described below.

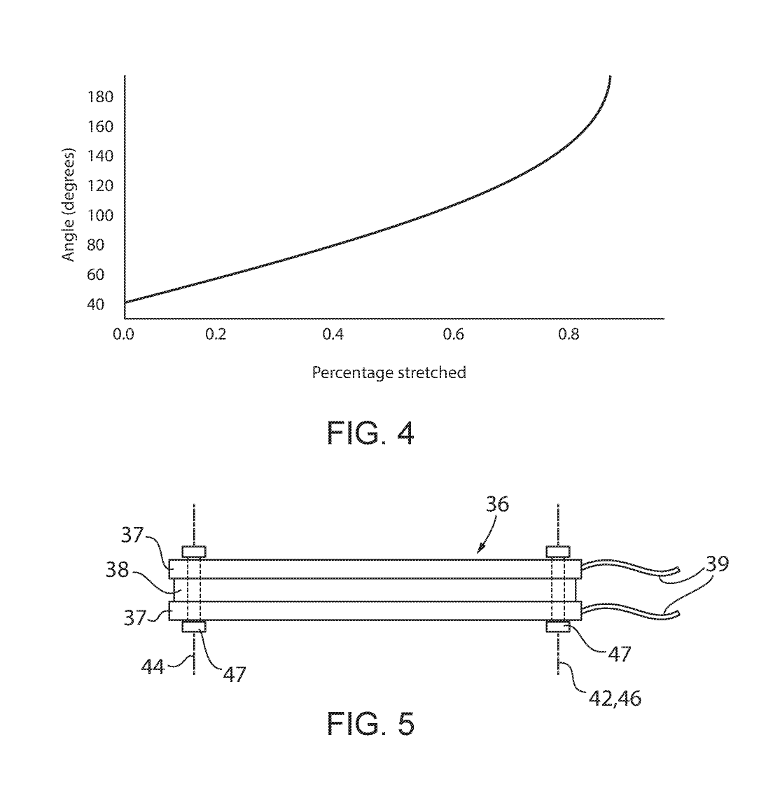

Referring now to FIGS. 2 and 4, the compressive strain related output data may be transmitted to the transmitter 40, which transmits the output data to an external computer 45 for further processing. The data may be saved to a memory 48 of the external computer 45 having a processor 50. The processor 50 may then process a program 52 stored in memory 48 to approximate an angle between the stationary arm 14 and the rotating arm 16 when given compressive strain related data as the input data for a “law of cosines” derived transfer function as further described below. Compressive strain related data from both EAP sensors 34, 35 may also be used to minimize nonlinearity in the transfer function.

In one embodiment of the present invention, the transfer function used to approximate the angle between the stationary arm 14 and the rotating arm 16 may be derived as described below:

The law of cosines may be simplified, as shown below, where Δ is the percentage stretch as a decimal, s is the length of the sensor, and x and y are the distance from the pivot axis 44 to the pivot axes 42, 46 of the EAP sensors 34, 35:

$$θ = {cos}^{- 1} ( \frac{- {( 10 + ( 1 + Δ ) s )}^{2} + {x}^{2} + {y}^{2}}{2 xy} ) 180 \text{/} π$$

To determine the location of the pivot axes 42, 46 for the EAP sensors 34, 35 the following equations are created with three unknowns (s, x and y).

$$s = \sqrt{{x}^{2} + {y}^{2} - 2 xy cos ( \frac{π}{6} )}$$

$$s = \frac{x + y}{1.9}$$

For example, by using 30° as the un-stretched case providing a 10° safety factor, and using the 180° case accounting for a 90% stretch, when s=50 mm, the EAP sensors 34, 35 yield the equation shown graphically in FIG. 4.

Based upon the transfer function described above, compressive strain related data, e.g., may be processed by the program 52 stored in memory 48 to approximate the angle, e.g., θ, between the stationary arm 14 and the rotating arm 16 of the range of motion measurement device 10, and therefore, representing the approximate angle between the patient's upper leg 24 and the patient's lower leg 26 and the angle of rotation θ of the patient's knee joint 11.

The two EAP sensors 34, 35 may be mounted such that each sensor 34, 35 reads in its linear range of the transfer function, and the combined range covers the full range of motion of the knee joint 11.

It is understood that the above example is not intended to limit the scope of the present invention in any way. Indeed, various modifications of the invention in addition to those shown and described herein will become apparent to those skilled in the art from the foregoing description and example and fall within the scope of the appended claims.

Certain terminology is used herein for purposes of reference only, and thus is not intended to be limiting. For example, terms such as “upper”, “lower”, “above”, and “below” refer to directions in the drawings to which reference is made. Terms such as “front”, “back”, “rear”, “bottom” and “side”, describe the orientation of portions of the component within a consistent but arbitrary frame of reference which is made clear by reference to the text and the associated drawings describing the component under discussion. Such terminology may include the words specifically mentioned above, derivatives thereof, and words of similar import. Similarly, the terms “first”, “second” and other such numerical terms referring to structures do not imply a sequence or order unless clearly indicated by the context.

When introducing elements or features of the present disclosure and the exemplary embodiments, the articles “a”, “an”, “the” and “said” are intended to mean that there are one or more of such elements or features. The terms “comprising”, “including” and “having” are intended to be inclusive and mean that there may be additional elements or features other than those specifically noted. It is further to be understood that the method steps, processes, and operations described herein are not to be construed as necessarily requiring their performance in the particular order discussed or illustrated, unless specifically identified as an order of performance. It is also to be understood that additional or alternative steps may be employed.

References to “a microprocessor” and “a processor” or “the microprocessor” and “the processor,” can be understood to include one or more microprocessors that can communicate in a stand-alone and/or a distributed environment(s), and can thus be configured to communicate via wired or wireless communications with other processors, where such one or more processor can be configured to operate on one or more processor-controlled devices that can be similar or different devices. Furthermore, references to memory, unless otherwise specified, can include one or more processor-readable and accessible memory elements and/or components that can be internal to the processor-controlled device, external to the processor-controlled device, and can be accessed via a wired or wireless network.

It is specifically intended that the present invention not be limited to the embodiments and illustrations contained herein and the claims should be understood to include modified forms of those embodiments including portions of the embodiments and combinations of elements of different embodiments as come within the scope of the following claims. All of the publications described herein, including patents and non-patent publications are hereby incorporated herein by reference in their entireties.

What we claim is:

1. A medical goniometer comprising:
a first patient support adapted to attach to a first side of a patient's joint;
a second patient support tangibly attached to the first patient support and hingably attached to an opposite second side of the patient's joint so that the first and second supports pivot about a hinge axis;
an elastomeric sensor providing electrical signal as a function of distortion of the sensor connected between the first patient support and the second patient support to be distorted by the pivoting; and
electrical leads attached to the elastomeric sensor to provide an output signal indicating an angle between the first patient support and second patient support.

2. The medical goniometer of claim 1 wherein the elastomeric sensor provides a change in capacitance with distortion of the elastomeric sensor.

3. The medical goniometer of claim 2 wherein the elastomeric sensor is two stretchable electrodes separated by an elastomer.

4. The medical goniometer of claim 3 wherein the two stretchable electrodes are carbon ink electrodes and the elastomer is a silicone dielectric polymer.

5. The medical goniometer of claim 3 wherein the electrical leads are attached to the elastomeric sensor at one end.

6. The medical goniometer of claim 1 wherein the elastomeric sensor comprises first and second sensors.

7. The medical goniometer of claim 6 wherein each of the first and second sensors is pivotably connected to the first patient support and second patient support about respective pivot axes.

8. The medical goniometer of claim 7 wherein the pivot axes and hinge axis are substantially aligned in parallel.

9. The medical goniometer of claim 8 wherein the first and second sensors have a shared pivot axis on the second patient support.

10. The medical goniometer of claim 1 wherein the distortion of the sensor is a change in length.

11. The medical goniometer of claim 1 wherein the first patient support and second patient support further comprising support straps adapted to wrap around the patient at the first side and second side of the patient's joint.

12. The medical goniometer of claim 1 further comprising a transmitter transmitting the output signal to a computer.

13. The medical goniometer of claim 12 wherein the computer is a mobile device.

14. An angle measurement device comprising:
a first support;
a second support pivotable with respect to the first support about a hinge axis and defining an angle of rotation between the first support and the second support;
a first elastomeric sensor providing electrical signal as a function of distortion of the sensor connected between the first support and the second support to be distorted by the pivoting;
a second elastomeric sensor providing electrical signal as a function of distortion of the sensor connected between the first patient support and the second patient support to be distorted by the pivoting; and
electrical leads attached to the elastomeric sensor to provide an output signal indicating an angle between the first support and second support.

15. The angle measurement device of claim 14 wherein each of the first and second sensors is pivotably connected to the first support and second support about respective pivot axes.

16. The angle measurement device of claim 15 wherein the first and second sensors have a shared pivot axis on the second support.

17. The angle measurement device of claim 14 further comprising a computer processor receiving the output signal and executing a stored program to output the angle of rotation between the first support and the second support.

18. The angle measurement device of claim 17 wherein the first support has a first section and a second section disposed at an angle wherein the first elastomeric sensor is attached to the first section and the second elastomeric sensor is attached to the second section.

19. The angle measurement device of claim 18 wherein the angle is between 130 and 140 degrees.

20. A method of measuring an angle of rotation comprising:
providing an angle measurement device having
a first patient support adapted to attach to a first side of a patient's joint;
a second patient support tangibly attached to the first patient support and hingably attached to an opposite second side of the patient's joint so that the first and second supports pivot about a hinge axis;
an elastomeric sensor providing electrical signal as a function of distortion of the sensor connected between the first patient support and the second patient support to be distorted by the pivoting; and
electrical leads attached to the elastomeric sensor to provide an output signal indicating an angle between the first patient support and second patient support;
attaching the first support to a first side of a patient's joint;
attaching the second support to a second side of the patient's joint; and
outputting the angle of rotation defined between the first support and the second support.
